SEQUENCE LISTING (31 sequences)

SEQ ID NO: 1 - DNA, 798 bp, Nosema ceranae
atggaattaatagcaacttttgcatttactttactatccttcggtttatgtagcattgaggatgctttaaaaggatcttgttttgataacagatctagattacattgtaatgatccgttcaattttaagcctagatgccattttgaattaagaagaccttgtattgaaactacgtgtatatttgatattgttgcagcagacaatgtaaagccatgcgaggctattagaatacagtgtataccatttaaagtacctagtacctgtaaagtagtacgaaaatatgttgaaaaaactcttgattacatcagatgtaaattgaacaaatgtgattaccaaaaaattaaatgcgatgctgaaatgattttaagaattatcgactgtaatctaaatggatgtcctaaggaatatgcgttatttaccgcatctgctttacacaacacttcatttttcaagtattttgcttcacactgtgacaaatctaaatacatgccaagaggacttttaatgattaataatatagctaattacaaaaaattggatgcagtaagtaaatataaaagaaactatgtgagatacccaacacgtctcgcgtgcaaattaaaagaagatattattaatacaattaggttttggaaatcgattatgtgcagaagacctatgacttttgcaggtatgatggatgctttaaatactgttagctggggaacttacaaacattctgatggaacttgtatgccagcagatttaaaaaatcgaaaagaaatttatgaggatttactcagacgatataaaaaataa

SEQ ID NO: 2 - DNA, 414 bp, Nosema ceranae
atgaataaagcaccaaaacaaaatacaggtaaagcgccaataagtactacacaagtcggagatctggacaaaaaaaagaagaagaaaatgtctaatttatgttctggtatgcaattcaaatctgcaataaaaaagattgccaaggatattaatcctgatcaaacagtatttttaactagtcaggccattgctattttatgtgctattgcttatgatattgtttctacttgtggtgaagtagcctctgagttttctaagaaagtaagtaaacagactgttggtgccgatgaaattgtggccgcatttgaaattttattaaaaggagaattaaagaaattagcattaagagaagtacacacttgtttgtccaagccaagtgcaccatctaagccttctaaatcccttaaagattaa

SEQ ID NO: 3 - DNA, 1128 bp, Nosema ceranae
atggaaaaaattactcaagctcttgttgttgacaatggttcaggtatcgtaaaagcaggatttgctggggatgatgctcctagagccgtatttccaagtattgttggtgtaccaagacacaagggtgtaatggtaggtatgggtcaaaaagatgcttatgtaggagatgaggcacaatctaaaagaggagtactagaacttcaatatcctattagtcatggtattgttacaaactggggtgatatggaaaagatctggcatcacactttttataatgaattaagggtttcaccagaggaacacccaattttattgactgaggcaccattaaatcctactgctaataaagaaaaaatggctcaggttatgtttgaaacttttaatgcccctcatttctatatttctattcaagctgttttatctctttacgctagtggaagaactaccggtattgtatttgactctggtgatggtgtctcccatgtggttccaatttatgaaggatattcattacctatggcaataagaaggcttaatttggctggtagagatttgactgagtacttacaagttattttaacagagagtggaaatagctttagtactactgctgaaaaagaaattgtaagagatattaaagaaaaattatgttatgtgtctgctgattatgaagaggatgttaagaataatagccttctttctagtattactaaaaattacgaaatgcctgatgggcaaattatttctattggaaatgaaaggtttagagcaccagaattattgtttaaaccacacttaagaggtttagaattaacaagtattcatgctactatctttgattctatcaaagcctgtgatgtggatattagaaaagatttatatgctaatattgtgttaagtggaggaacaactttgttcccaggtattaatgttagattagatagggaaatcagaaacttggcaccaagctcagttaaaattaatgttgtaacacctgctgaaagaaaatacagtgtatggattggtggatcaattgttgcttcactttcaacattccaagaaatgtgggtatctaaacaagattatcaagaatatggaccacctattgtcaacagaaaatgcttttaa

SEQ ID NO: 4 - DNA, 1023 bp, Nosema ceranae
atgaatacatcacaaaaaaaactgcttaaattacaattaaatcctaattatagacatctttattattattattttttttgtttacatttattagttttatttattaattccttttttattagccccatgaacagttcaaatagtaaatttataaatgttttaaacttttggaaagctttagaaggaagtaaaaaagttgaaatgccgaagaatgatgtacttttagtgccttgtgatgcaaaaattatacaaaaagataatgtatttgtaattggtagtaatgaagaccttcttcaaaaagataatttaatagtgaaattcaaagataataaacttactaaaaatgatgatgcaattgtaaaattaaatgattgtaaattaacaaaaaaagatgacacgatcgtgactttaaatgagtgtaagatatttgaaaaaagcgatataattgtacaactaattgatattcagtttactagaaaaaaaatattttttactaaagaatttattaatgacttgaacaacatttatattagaagaatgttaccgacgcgtagtgaaaaaaaatttatacgaagcttaatgtcatgtagtattgatgatattacaagcgaggaatctgacacaatacacgaaactatttcacataaaaagttcagtgataatcaaactacatatgatatcaaaaatacagacgatagaacagatgatgattttaattcgatatttgaattttattacaacagtgacacaaataaatcagatactgatcagataaataatttaagtcaagaaaaatgcaagacggattttaataaagtaaacaatcacaaaaaagtaacctttaaagatccttatgaagaatatgatgagggattaaatgatgaattccccttattaataaatataatgaaggcagaatctgaagggtatgtagcgaaagaattggcaaaaaagaagaaaaaagggtggtttcaaagatttaaagaattgttttgttgtttatgtagcgacaatgtatattaa

SEQ ID NO: 5 - DNA, 20 bp, Artificial Sequence
  Primer
attaaatgcgatgctgaaat

SEQ ID NO: 6 - DNA, 20 bp, Artificial Sequence
  Primer
aatctgctggcatacaagtt

SEQ ID NO: 7 - DNA, 20 bp, Artificial Sequence
  Primer
tgaataaagcaccaaaacaa

SEQ ID NO: 8 - DNA, 21 bp, Artificial Sequence
  Primer
ggatttagaaggcttagatgg

SEQ ID NO: 9 - DNA, 21 bp, Artificial Sequence
  Primer
acaatggttcaggtatcgtaa

SEQ ID NO: 10 - DNA, 20 bp, Artificial Sequence
  Primer
tcaggcatttcgtaattttt

SEQ ID NO: 11 - DNA, 20 bp, Artificial Sequence
  Primer
ttattagccccatgaacagt

SEQ ID NO: 12 - DNA, 20 bp, Artificial Sequence
  Primer
gcgtcggtaacattcttcta

SEQ ID NO: 13 - DNA, 18 bp, Artificial Sequence
  Primer
caccaggttgattctgcc

SEQ ID NO: 14 - DNA, 21 bp, Artificial Sequence
  Primer
ttatgatcctgctaatggttc

SEQ ID NO: 15 - DNA, 24 bp, Artificial Sequence
  Primer
tgccttatcagctntcgattgtag

SEQ ID NO: 16 - DNA, 24 bp, Artificial Sequence
  Primer
ttcagntttgcaaccatacttccc

SEQ ID NO: 17 - DNA, 20 bp, Artificial Sequence
  Primer
tcctcaagcttggaaaagag

SEQ ID NO: 18 - DNA, 20 bp, Artificial Sequence
  Primer
ggtggacaaagaagcaagaa

SEQ ID NO: 19 - DNA, 22 bp, Artificial Sequence
  Primer
ctaatacgactcactatagggc

SEQ ID NO: 20 - DNA, 22 bp, Artificial Sequence
  Primer
tcgagcggccgcccgggcaggt

SEQ ID NO: 21 - DNA, 20 bp, Artificial Sequence
  Primer
agcgtggtcgcggccgaggt

SEQ ID NO: 22 - DNA, 17 bp, Artificial Sequence
  Primer
gttttcccagtcacgac

SEQ ID NO: 23 - DNA, 22 bp, Artificial Sequence
  Primer
tcacacaggaaacagctatgac

SEQ ID NO: 24 - DNA, 798 bp, Nosema ceranae; identical to SEQ ID NO: 1

SEQ ID NO: 25 - DNA, 414 bp, Nosema ceranae; identical to SEQ ID NO: 2

SEQ ID NO: 26 - DNA, 1128 bp, Nosema ceranae; identical to SEQ ID NO: 3

SEQ ID NO: 27 - DNA, 1023 bp, Nosema ceranae; identical to SEQ ID NO: 4

SEQ ID NO: 28 - DNA, 406 bp, Nosema ceranae
attaaatgcgatgctgaaatgattttaagaattatcgactgtaatctaaatggatgtcctaaggaatatgcgttatttaccgcatctgctttacacaacacttcatttttcaagtattttgcttcacactgtgacaaatctaaatacatgccaagaggacttttaatgattaataatatagctaattacaaaaaattggatgcagtaagtaaatataaaagaaactatgtgagatacccaacacgtctcgcgtgcaaattaaaagaagatattattaatacaattaggttttggaaatcgattatgtgcagaagacctatgacttttgcaggtatgatggatgctttaaatactgttagctggggaacttacaaacattctgatggaacttgtatgccagcagatt

SEQ ID NO: 29 - DNA, 401 bp, Nosema ceranae
tgaataaagcaccaaaacaaaatacaggtaaagcgccaataagtactacacaagtcggagatctggacaaaaaaaagaagaagaaaatgtctaatttatgttctggtatgcaattcaaatctgcaataaaaaagattgccaaggatattaatcctgatcaaacagtatttttaactagtcaggccattgctattttatgtgctattgcttatgatattgtttctacttgtggtgaagtagcctctgagttttctaagaaagtaagtaaacagactgttggtgccgatgaaattgtggccgcatttgaaattttattaaaaggagaattaaagaaattagcattaagagaagtacacacttgtttgtccaagccaagtgcaccatctaagccttctaaatcc

SEQ ID NO: 30 - DNA, 700 bp, Nosema ceranae
acaatggttcaggtatcgtaaaagcaggatttgctggggatgatgctcctagagccgtatttccaagtattgttggtgtaccaagacacaagggtgtaatggtaggtatgggtcaaaaagatgcttatgtaggagatgaggcacaatctaaaagaggagtactagaacttcaatatcctattagtcatggtattgttacaaactggggtgatatggaaaagatctggcatcacactttttataatgaattaagggtttcaccagaggaacacccaattttattgactgaggcaccattaaatcctactgctaataaagaaaaaatggctcaggttatgtttgaaacttttaatgcccctcatttctatatttctattcaagctgttttatctctttacgctagtggaagaactaccggtattgtatttgactctggtgatggtgtctcccatgtggttccaatttatgaaggatattcattacctatggcaataagaaggcttaatttggctggtagagatttgactgagtacttacaagttattttaacagagagtggaaatagctttagtactactgctgaaaaagaaattgtaagagatattaaagaaaaattatgttatgtgtctgctgattatgaagaggatgttaagaataatagccttctttctagtattactaaaaattacgaaatgcctga

SEQ ID NO: 31 - DNA, 435 bp, Nosema ceranae
ttattagccccatgaacagttcaaatagtaaatttataaatgttttaaacttttggaaagctttagaaggaagtaaaaaagttgaaatgccgaagaatgatgtacttttagtgccttgtgatgcaaaaattatacaaaaagataatgtatttgtaattggtagtaatgaagaccttcttcaaaaagataatttaatagtgaaattcaaagataataaacttactaaaaatgatgatgcaattgtaaaattaaatgattgtaaattaacaaaaaaagatgacacgatcgtgactttaaatgagtgtaagatatttgaaaaaagcgatataattgtacaactaattgatattcagtttactagaaaaaaaatattttttactaaagaatttattaatgacttgaacaacatttatattagaagaatgttaccgacgc

(19) United States
(12) Patent Application Publication
(10) Pub. No.: US 2015/0038353 A1
(43) Pub. Date: Feb. 5, 2015

(54) MOLECULAR MARKERS FOR DETECTING NOSEMA INFECTION

(71) Applicant: National Taiwan University, Taipei City (TW)

(72) Inventors: Chu-Fang LO, Taipei City (TW); Chung-Hsiung WANG, Taipei City (TW); Yu-Shin NAI, Taipei City (TW)

(73) Assignee: National Taiwan University, Taipei City (TW)

(21) Appl. No.: 14/265,422

(22) Filed: Apr. 30, 2014

(30) Foreign Application Priority Data
Aug. 1, 2013 (TW) 102127588

(51) Int. Cl. C12Q 1/68; C07K 14/37

(52) U.S. Cl. CPC C12Q 1/6895; C07K 14/37; C12Q 2600/16
USPC 506/9; 536/23.74; 435/6.12; 435/6.11; 536/24.32; 536/24.33

(57) ABSTRACT

The present invention provides a molecular marker for detecting Nosema infection, comprising at least one sequence selected from a gene sequence or a complementary sequence thereof of SR-22, SR-28, SR-71, and SR-85. By designing specific primer sets based on the sequences of molecular marker, the early stage and latent infections of Nosema can be detected, and the infection levels can also be determined.

### RELATED APPLICATIONS

This application claims priority to Taiwan Application Serial Number 102127588, filed Aug. 1, 2013, which is incorporated by reference herein in its entirety.

### BACKGROUND

1. Technical Field

The present invention is related to a molecular marker for detecting Nosema infection; focused on Nosema infection detection and infection level determination.

2. Description of Related Art

Colony Collapse Disorder (CCD) was defined as the phenomenon of disappearing honeybees in bee colonies. The phenomenon occurs worldwide and also similar cases are noted in Taiwan recently. According to the previous researches (http://sciscape2.org/2011/02/10/13759.htm# more-13759), honeybee colonies significantly disappeared and the cultivated bee colonies also decreased from 1971 to 2006, and even until now. The honeybees play a major role on pollination that is extremely important for the agriculture ecosystem. Therefore, CCD is now an important scientific issue.

Although some evidences pinpointed the possible reason for CCD that is caused by cross infection of virus and fungus, it still requires further researches to clarify. Many scientists believe that Nosema Disease is one of the major factors. Traditionally, the detection of Nosema infection is dependent on light microscopic examination of spores existed in the feces and/or midgut of honeybees. However, spore formation is not occurring at the early stage of the infection, therefore the microscopic examinations will difficult to detect the infection at early stage and even at latency stage The genome of Nosema has multiple repeats of SSUrDNA; therefore, to detect the infection via examining SSUrDNA by PCR is sensitive. The method is useful in detecting the infection at early or latency stages and is favorable for prevention of Nosema infection. However, in spite of the fact that the SSUrDNA is suitable for early detection, this marker might be limited to determine the infection level of this parasite, i.e. the copy number of this parasite in honeybee midgut.

In light of the foregoing, CCD may result in imbalance of the ecosystem and brings disaster to our world. The conventional detection methods for Nosema infection are not ideal. Therefore, it continuously requires a detection method not only for detection at the early and latency infection stages but also for determination of the infection levels.

### SUMMARY

One of the objects of the present invention is to provide a molecular marker and a kit containing the same for detecting Nosema infection at early and latency stages.

Another object of the present invention is to provide a method for detecting Nosema infection, which can detect the infection at early stage and is capable of tracing the infection level.

In order to achieve the aforesaid objects, the present invention provides a molecular marker for detecting Nosema infection, comprising at least one sequence selected from a group consisting of DNA sequences of SR-22, SR-28, SR-71, and SR-85, cDNA sequences thereof, fragments thereof, and complementary sequences thereof.

Preferably, said molecular marker comprises at least one sequence selected from a group consisting of SEQ ID NO 01, SEQ ID NO 02, SEQ ID NO 03, SEQ ID NO 04, SEQ ID NO 24, SEQ ID NO 25, SEQ ID NO 26, SEQ ID NO 27, SEQ ID NO 28, SEQ ID NO 29, SEQ ID NO 30, and SEQ ID NO 31.

Preferably, said molecular marker further comprises a SSUrDNA sequence of Nosema.

The present invention also provides a method for detecting is Nosema infection, comprising: (A) obtaining a genetic material; (B) obtaining a nucleic acid, having at least parts of its sequences being complementary to the molecular marker of claim 1 or claim 2; (C) base-pairing said nucleic acid with said genetic material and examining the result of said base-pairing.

Preferably, said genetic material is deoxyribonucleic acid, ribonucleic acid, or a combination thereof.

Preferably, said nucleic acid is deoxyribonucleic acid, ribonucleic acid, or a combination thereof.

Preferably, said nucleic acid is a primer set, a probe, or a combination thereof.

Preferably, said primer set comprising: a primer set of SEQ ID NO 05 and SEQ ID NO 06, a primer set of SEQ ID NO 07 and SEQ ID NO 08, a primer set of SEQ ID NO 09 and SEQ ID NO 10, or a primer set of SEQ ID NO 11 and SEQ ID NO 12.

Preferably, said step (c) is achieved by polymerase chain reaction (PCR), reverse-transcription polymerase chain reaction (RT-PCR), real-time polymerase chain reaction (Real-time PCR), dot blotting, or a combination thereof.

Preferably, said method comprises detecting the existence of SSUrDNA of Nosema in said genetic material.

Preferably, said detecting Nosema infection comprises detecting the infection level.

The present invention also provides a kit for detecting Nosema infection, comprising: a nucleic acid, having at least parts of is its sequences being complementary to the aforesaid molecular marker.

Preferably, said nucleic acid is deoxyribonucleic acid, ribonucleic acid, or a combination thereof.

Preferably, said nucleic acid is a primer set, a probe, or a combination thereof.

Preferably, said primer set comprising: a primer set of SEQ ID NO 05 and SEQ ID NO 06, a primer set of SEQ ID NO 07 and SEQ ID NO 08, a primer set of SEQ ID NO 09 and SEQ ID NO 10, or a primer set of SEQ ID NO 11 and SEQ ID NO 12.

To sum up, the present invention is related to a molecular marker for detecting Nosema infection and is related to a detection method of using said molecular marker as well as a kit containing the same. The present molecular marker can be used in the common molecular detection technologies for Nosema infection at early and/or latency stages. Moreover, the present molecular marker is capable of distinguishing the infection level; therefore is particularly favorable for tracing the spread of this disease and setting the time table for preventing huge loss in bee colonies.

### BRIEF DESCRIPTION OF THE DRAWINGS

FIG. 1 shows the result of microscopic examinations of the Example 2 of the present invention.

FIG. 2 shows the result of SSUrDNA detection of the Example 2 of the present invention.

FIG. 3 shows the result of the present method of the Example 2 of the present invention.

### DETAILED DESCRIPTION

Nosema Disease is a common disease of honeybees, and scientists consider it to be one of the factors causing Colony Collapse Disorder (CCD). Therefore, a method capable of detecting Nosema infection at early and/or latency stages shall be useful for prevention of CCD.

In one embodiment, the motivation of present invention is to discover a molecular marker that is specific for Nosema. Preferably, this molecular marker with the genomic analysis technology can be used for Nosema infection detection. On the other hand, the concept of the present invention is to take the molecular technologies' advantage of high sensitivity for Nosema infection detection by using molecular markers that are specific to Nosema. At the gene level, common molecular technologies used for examining or quantifying gene expression include but not limited to PCR, RT-PCR, Real-time PCR, and dot blotting method. Usually, those technologies need only a small amount of sample for conducting the detection; therefore they provide the possibility of detecting the target at early stage. On the other hand, those technologies are capable of quantification; therefore can estimate the infection level. Four Nosema genes, SR-22, SR-28, SR-71, and SR-85, are screened by Suppression Subtractive Hybridization (SSH) in the present invention and proved to be to potential specific markers for Nosema infection and even for the infection level. The present molecular marker for detecting Nosema infection comprises at least one sequence selected from the DNA sequences of SR-22, SR-28, SR-71, and SR-85, cDNA sequences thereof, fragments thereof, and complementary sequences thereof. The is experiments of the present invention proved that SR-22, SR-28, SR-71, and SR-85 can specifically represent the occurrence of Nosema infection. Those having ordinary skill in the art can appreciate that a DNA or RNA has counterparts that substantially base-pairing with, hybridizing with, or forming hydrogen bonding with. Thus, the term “complementary sequences thereof” recited in the instant specification means sequences that can form base-paring while using said SR-22, SR-28, SR-71, or SR-85 as a template.

More specifically, the present molecular marker comprises at least one sequence selected from a group consisting of SEQ ID NO 01, SEQ ID NO 02, SEQ ID NO 03, and SEQ ID NO 04, and complementary sequences thereof. Besides, those having ordinary skill in the art shall appreciate that the cDNA of said genes or any gene fragment sufficiently representing said genes are also operable as the molecular marker of the present invention. Alternatively, said molecular marker can also include the tools that have been commonly used for detecting Nosema infection, such as, SSUrDNA.

In another embodiment, the present invention provides a method for detecting Nosema infection by using the molecular markers as above. Briefly, the first step of our method is to obtain the genetic material of the samples to be examined. The genetic material may be the DNA, RNA or a combination thereof of the object. For instance, the total DNA obtained from the feces or midgut of honeybees can be used for the following steps.

Then, a nucleic acid is artificially designed for targeting molecular marker as above; wherein said nucleic acid shall be at least partially complementary to parts of the present molecular marker. Said nucleic acid may be deoxyribonucleic acid, ribonucleic acid, or a combination thereof. It is no need to limit the design method as there are various design methods is known in the field and there are lots of online database available for designing said nucleic acid. Said nucleic acid may be a primer set, a probe, or a combination thereof. The primer set used in the present invention are listed in the following table 1:

TABLE 1

| Name | Sequence (5′ to 3′) | SEQ ID NO |
|---|---|---|
| RT-SR22F | ATT AAA TGC GAT GCT GAA AT | SEQ ID NO 05 |
| RT-SR22R | AAT CTG CTG GCA TAC AAG TT | SEQ ID NO 06 |
| RT-SR28F | TGA ATA AAG CAC CAA AAC AA | SEQ ID NO 07 |
| RT-SR28R | GGA TTT AGA AGG CTT AGA TGG | SEQ ID NO 08 |
| RT-SR71F | ACA ATG GTT CAG GTA TCG TAA | SEQ ID NO 09 |
| RT-SR71R | TCA GGC ATT TCG TAA TTT TT | SEQ ID NO 10 |
| RT-SR85F | TTA TTA GCC CCA TGA ACA GT | SEQ ID NO 11 |
| RT-SR85R | GCG TCG GTA ACA TTC TTC TA | SEQ ID NO 12 |

After that, base-pairing between said nucleic acid and said genetic material is performed. Said “base-pairing” is referred as a process that hydrogen bonding is formed between the bases of said nucleic acid and the bases of said molecular marker under certain condition; more specifically, said “base-pairing” includes the annealing step in a PCR and the hybridization step in a dot blotting. It is unnecessary to limit said “certain condition” as the user can adjust for proper temperature, reaction time and reaction cycles based on the molecular technologies and the nucleic acid used.

Lastly, the result of said base-pairing is examined for detecting the existence of said molecular marker. Said “examining the result of said base-pairing” is referred as confirming if said base-pairing is happened and/or confirming the extent of said base-pairing. Said “examining” can be achieved by any method known in the art, for instance, some types of fluorescent proteins can be used in the base-pairing (ex. SYBR green). Those types of fluorescent proteins can release detectable fluorescence while a double stranded nucleic acid is formed from the base-pairing of said nucleic acid and said molecular marker. By using this concept, the result of said base-pairing can be examined by detecting the fluorescence; moreover, the infection level can be estimated by quantifying the amount of fluorescence.

By referring to the disclosure of the present invention, those having ordinary skill in the art can easily understand several operable manners for conducting the step (C) of the present method, such as PCR, RT-PCR, real-time PCR, and dot blotting. Taking RT-PCR as an example, the PCR product can be applied for electrophoresis and the result of electrophoresis can be observed under UV light for examining the base-pairing after stained with EtBr.

It is important to note that, from the preceding paragraphs, the “nucleic acid” and the following “examining the result of said base-pairing” are co-related features of the present invention. That is, once the manner for “examining the result of said base-pairing” is decided and the type of said “nucleic acid” shall be correspondingly limited. If said “examining the result of said base-pairing” is conducted by detecting the amount of fluorescence, then said “nucleic acid” shall be modified with is fluorescent material or some types of fluorescent materials shall participate in the base-pairing reaction (such as, Real-time PCR). In addition, if said “examining the result of said base-pairing” is conducted by PCR-based technologies, then said “nucleic acid” shall be a primer set; whereas, if said “examining the result of said base-pairing” is conducted by dot blotting, then said “nucleic acid” shall be a probe tagged with radioactive substance.

The intensity of the detection signal of conventional molecular marker, such as the SSUrDNA of Nosema, is easily to be saturated as the genome of Nosema has multiple repeats of SSUrDNA. Distinguishing the infection level is impossible while using SSUrDNA as a molecular marker. Whereas, the present molecular marker is characterized that not only the infection of Nosema can be detected but also the infection level can be distinguished. As mentioned above, the infection level can be recognized from the amount of fluorescence or by comparison of the intensity between two samples under UV light.

In yet embodiment, the present invention provides a kit for detecting Nosema infection, which comprises a nucleic acid having at least parts of its sequences being complementary to the present molecular marker. From the view of commercialization, the kit of the present invention can be designed in an easier-to-use way. For instance, said “examining the result of said base-pairing” can be conducted by a color reaction; therefore, it will be more convenient for user to examine the infection and level thereof by observation. Said color reaction has been applied in various experimental or household detection devices and been well-known in the art. The key feature of present kit is containing a nucleic acid having at least parts of its sequences being complementary to the present molecular marker; thus, those having ordinary skill in the art may modify the feature by any known manner in the art in order to make the kit more suitable for commercialization (including, suitable for large-scale production or convenient to identify the detection result) but still within the scope of the present invention.

The following embodiments are recited for further explaining the advantages of the present invention but not for limiting the claim scope of the present invention.

### Example 1

### Screening to Obtain the Molecular Marker of the Present Invention by Suppression Subtractive Hybridization

Honeybee pupae were placed in 24-well plates and kept in an incubator at 30±2° C. to provide newly emerged Nosema free honeybees. Three days after eclosion, the bees were starved for 2 h before infection. For Nosema infecting, the bees were fed with 5 μl of 50% sucrose solution containing 1.25×10⁵ spores of N. ceranae by a droplet with the spore solution at the tip of a micropipette until it had consumed the entire droplet

### [RNA Extraction]

At the 7, 14 and 21 days post infection, the mid-guts of infected bees and healthy bees were collected into centrifuge tubes. Total RNA was extracted using TRIzol® Reagent (ambion) according to the manufacturer's instructions. The tissues were homogenated by sterile tissue grinder. Then, the tubes were vortexed for 15 seconds to homogenate the tissues. The tubes were iced for 15 minutes and chloroform was added at the ratio of 0.2 ml/1 ml. The tubes were vortex again for 15 seconds. After that, the upper water-layer was transferred into clean centrifuge tubes and 2-propenol of equal volume was added. After iced for 15 minutes, the tube was centrifuged at 14,000×g (4° C.) for 15 minutes. Then, the supernatant was is discarded and the sediment was collected.

The ethanol (70%) was added to wash extra ions in the sediment and the mixture was centrifuged at 14,000×g (4° C.) for 15 minutes. The centrifugation step was repeated one time and the sediment was placed in a laminar flow to dry out the remained ethanol. Lastly, the sediment was dissolved in 30-50 μl of sterile water treated with diethyl pyocarbonate (DSPC) and a RNA solution for the following experimental steps was obtained. The RNA solution was then examined by spectrophotometer for qualification and quantification.

[cDNA Synthesis]

The eDNA synthesis was generated by SMARTer™ PCR cDNA Synthesis Kit. Briefly, 1 μg of previously obtained RNA was mixed with 3′SMART CDS Primer II A (12 μM) cDNA synthesis primers of the kit and reacted in a PCR machine under the following program: 72° C. for 3 minutes and 42° C. for 2 minutes. Then, SMARTScribe reverse transcriptase, 5×first-stranded buffer, DTT (100 mM), dNTP mix (10 mM), SMARTer II A Oligonucleotide)(12 μM) and RNase Inhibitor (40 U/μl) were added. The mixture was for reverse-transcription at 42° C. for 1.5 hours and heated at 70° C. for extra 10 minutes to cease the reaction. Then, according to the manual, TE buffer was added and the first strand of cDNA was obtained.

The second strand cDNA was generated by PCR. A mixture of ss cDNA of proper amount, 5′PCR primer II A (12 μM), polymerase mix, 10×PCR buffer, 50×dNTP mix, and ddH₂O was set for PCR under the is following program: 95° C. for 1 minute, 95° C. for 15 seconds, 65° C. for 30 seconds, 68° C. for 6 minutes. The program was respectively repeated for 15, 18, 21, 24, and 27 cycles. The PCR products were examined by electrophoresis for determining which repeating cycle has the best synthesis efficiency. Then, based on the electrophoresis result, the second strand cDNA was prepared and a double-stranded cDNA (ds cDNA) of the most yield rate was obtained. Phenol/chloroform/isoamyl alcohol (25:24:1) was added for purifying the ds cDNA. After removing the water content, the re-suspended ds cDNA solution was passed through CHROMA SPIN™-1000+DEPC-H₂O column and obtain a purified ds cDNA for Suppression Subtractive Hybridization.

### [Suppression Subtractive Hybridization]

The cDNA from the infected honeybees was used as the tester (forward subtractive cDNA library, fscl.)/driver (reverse subtractive cDNA library, rscl.), and cDNA from the control honeybees as the driver (fscl.)/tester (rscl.). Both tester and driver cDNAs were digested with RsaI to produce shorter bluntended fragments. After digestion with RsaI, the tester cDNA was divided into two portions, each of which was ligated with a different adapter (Adaptor-1 and Adaptor-2R) at 16° C. for overnight. After ligation, each tester cDNA was separately hybridized at 68° C. for 8 h with an excess of driver cDNA after denaturation at 98° C. for 90 see. Then the two hybridized samples were mixed together and hybridized at 68° C. for overnight with excess of denatured driver cDNA. The resulting mixture was added with 200 ml dilution buffer and amplified by two rounds of suppression PCR. Before the primary PCR, the reaction mixture was incubated at 75° C. for 5 min to extend the adaptors. Primary PCR was performed at 94° C. for 30 sec, 66° C. for 30 sec, and 72° C. for 90 sec for 27 cycles in a reaction volume of 25 μl with PCR Primer 1 (SEQ ID NO 19): 5′-CTAATACGACTCACTA TAGGGC-3′. The PCR product was then diluted 10-fold for nest PCR. The nest PCR primer 1/2R (nested PCR primer 1 (SEQ ID NO 20): 5′-TCGAGCGGCCGCCCGGGCAGGT-3′/nested PCR primer 2R (SEQ ID NO 21):5′-AGCGTGGTCGCGGCCGAGGT-3′) were used for nest PCR. The subtracted secondary PCR products were ligated into T & A cloning vector (RBC) and transformation into DH5α competent cells to generate subtracted eDNA libraries.

Colonies were picked based on blue and white screening and a colony PCR using a primer set of M13F: 5′-GTTTTCCCAGTCACGAC-3′(SEQ ID NO 22)/M13R: 5′-TCACACAGGAAACAGCTATGAC-3′ (SEQ ID NO 23) targeting the T&A plasmid was conducted to confirm that the picked colonies did have the inserted DNA fragment (100˜1000 bp). The picked colonies were cultured and stored in glycerol at −80° C. and prepared for sequencing.

### [Analysis for the Differently-Expressing Genes]

cDNA sequences from the each SSH libraries were sequenced. CodonCode Aligner was used for base-calling (Q>13), trimming vector sequences, sequences assemblage. All the sequences were filtered for size (less than 100 bp). Putative functions of the unique sequences were discovered by using tBLASTn to translate each nucleotide query sequence into all reading frames and then searching for matches in the NCBI non-redundant database. Lastly, the results were compared with the UniProt database, which provides value-added information reports for protein functions. The UniProt reports consist of Gene Ontology (GO) annotations that classify proteins by biological process, cellular component, is and molecular function. Each unique sequence was tentatively assigned GO classification based on annotation of the single “best hit” match in UniProt. These data were then used to classify the corresponding genes according to their GO functions.

This example screened out SR-22, SR-28, SR-71, and SR-85 as potential molecular markers, which respectively have the following sequences: SEQ ID NO 01, SEQ ID NO 02, SEQ ID NO 03, and SEQ ID NO 04; moreover, their cDNA sequences are: SEQ ID NO 24, SEQ ID NO 25, SEQ ID NO 26, and SEQ ID NO 27. The following experiments were conducted to examine the application of those molecular markers in detecting Nosema infection.

### Example 2

### Detection of Nosema infection

Bee pupae before eclosion were placed in 24-well plate and kept in an incubator at 30±2° C. to provide newly emerged Nosema free bees. After eclosion, the bees were fed with 50% sugar solution for three days. Then, the bees were starved for 2 hours before infection. For infecting the bees with Nosema, the bees were fed with sugar solution containing fixed amount of Nosema. The average Nosema infection amount for each bee is 1.25×10⁵ spores/5 μl.

### [Microscopic Examination]

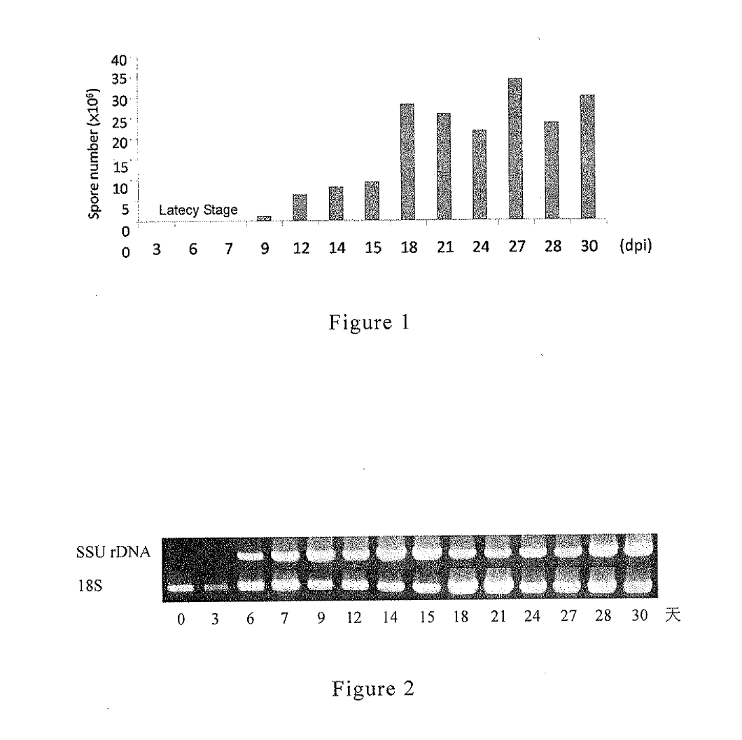

At the 3, 6, 7, 9, 12, 14, 15, 18, 21, 24, 27, 28 and 30 days post infection, the bees were dissected to obtain the mid-guts thereof (n=3) and the obtained mid-guts in 500 μl of TE buffer (10 mM Iris, 1 mM EDTA, pH 7.5) were homogenate by tissue grinder. The amount of mature spores was recorded by haemocytometer under phase-contrast microscopy (Olympus IX71). The data was showed as average amount of spores vs. day post infection (DPI) and displayed as FIG. 1.

### [SSUrDNA Assay]

As mentioned above, the mid-guts of the bees were obtained at several time points after being infected and homogenated by tissue grinder in 500 μl of TE buffer (10 mM Tris, 1 mM EDTA, pH 7.5). The mixtures were then treated with RNase A at 37° C. for 1 hour and then with proteinase K at 56° C. for 16 hours. An equal volume of phenol-chloroform-isoamyl alcohol (25:24:1) was added and the mixtures were centrifuged at 4° C. for 10 minutes. After centrifugation, the water layer was collected, a solution, containing 3M sodium acetate of 1/10 volume and ethanol of 2× volume, was then added. A DNA pellet was obtained by ethanol precipitation. Then, the DNA pellet was re-dissolved in ddH₂O of proper volume to obtain a DNA solution for the following steps.

A PCR was conducted for the SSUrDNA of the aforesaid generic material (the DNA solution). The primer set used are listed in the Table 2.

TABLE 2

| Name | Sequence (5′ to 3′) | SEQ ID NO |
|---|---|---|
| SSUrDNA 18f | CAC CAG GTT GAT TCT GCC | SEQ ID NO 13 |
| SSUrDNA 1537r | TTA TGA TCC TGC TAA TGG TTC | SEQ ID NO 14 |
| Inner Control (18S) 143F | TGC CTT ATC AGC TNT CGA TTG TAG | SEQ ID NO 15 |
| Inner Control (18S) 145R | TTC AGN TTT GCA ACC ATA CTT CCC | SEQ ID NO 16 |

The PCR condition was:

Total volume of the mixture was 50 μl containing 5 μl 10× reaction buffer (Bioman)·4 μl 2.5 mM dNTPs·0.5 μl 100 mM of the aforesaid primer sets·1 μl 1.25 U HiFi Taq polymerase (RBC) and 1 μl of DNA solution.

PCR program was performed by 35 cycles, each cycle was 95° C. for 5 minutes, 94° C. for 30 seconds, 50° C. for 1 minute, and 72° C. for 2 minutes. Finally, an extension period of 72° C. was done for 10 minutes.

The PCR product was examined by electrophoresis and observed under UV light after being stained by EtBr. The result is showed in FIG. 2.

### [Method of the Present Invention]

Total RNAs of the middle guts of the infected and healthy bees were collected at 7, 14, and 21 days post infection according, to the method described in the Example 1. cDNA solutions thereof were also prepared in accordance with Example 1. Then a reverse transcription PCR was conducted by using the primer sets listed in the following Table 3.

TABLE 3

| Name | Sequence (5′ to 3′) | SEQ ID NO |
|---|---|---|
| RT-SR22F | ATT AAA TGC GAT GCT GAA AT | SEQ ID NO 05 |
| RT-SR22R | AAT CTG CTG GCA TAC AAG TT | SEQ ID NO 06 |
| RT-SR28F | TGA ATA AAG CAC CAA AAC AA | SEQ ID NO 07 |
| RT-SR28R | GGA TTT AGA AGG CTT AGA TGG | SEQ ID NO 08 |
| RT-SR7IF | ACA ATG GTT CAG GTA TCG TAA | SEQ ID NO 09 |
| RT-SR71R | TCA GGC ATT TCG TAA TTT TT | SEQ ID NO 10 |
| RT-SR85F | TTA TTA GCC CCA TGA ACA GT | SEQ ID NO 11 |
| RT-SR85R | GCG TCG GTA ACA TTC TTC TA | SEQ ID NO 12 |
| Bee | TCC TCA AGC TTG GAA AAG AG | SEQ ID NO 17 |
| actin-f | | |
| Bee | GGT GGA CAA AGA AGC AAG AA | SEQ ID NO 18 |
| actin-r | | |

The PCR condition:

Total volume of the mixture was 50 μl containing 5 μl 10× reaction buffer (Bioman)·4 μl 2.5 mM dNTPs·0.5 μl 100 mM of the aforesaid primer sets·1 μl 1.25 U HiFi Taq polymerase (RBC) and 1 μl of cDNA solution.

PCR program was performed by 30 cycles, each cycle was 95° C. for 5 minutes, 94° C. for 30 seconds, 50° C. for 30 seconds, and 72° C. for 2 minutes. Finally, an extension period of 72° C. was done for 2 minutes.

The PCR product was examined by electrophoresis and observed under UV light after being stained by EtBr. The result is showed in Figure to 3. Besides, the PCR products of SEQ ID NO 05/SEQ ID NO 06 primer set, SEQ ID NO 07/SEQ ID NO 08 primer set, SEQ ID NO 09/SEQ ID NO 10 primer set, and SEQ ID NO 11/SEQ ID NO 12 primer set were sequenced to confirm they are respectively fragments of SR-22, SR-28, SR-71, and SR-85 genes, these experiments detected indeed the present molecular markers and the experimental results were not due to non-specific binding.

### [Comparison]

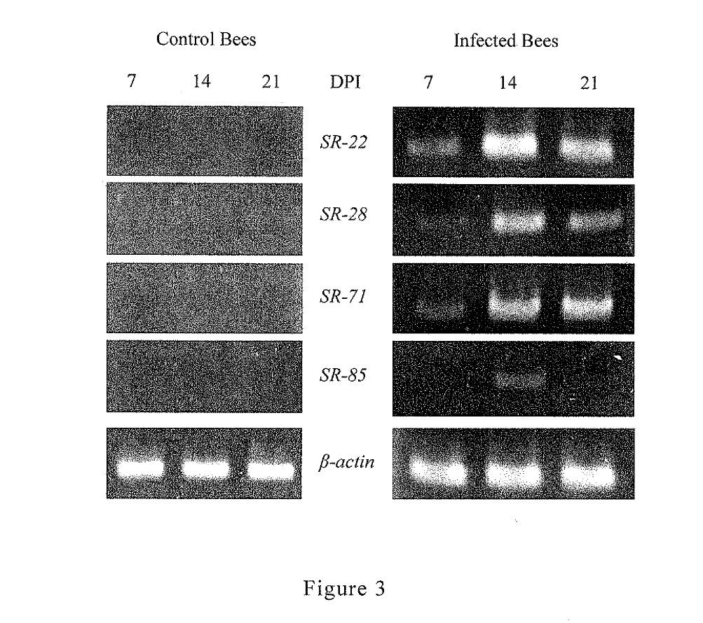

By comparing the data showed in FIG. 1, FIG. 2 and FIG. 3, it is noted that Microscopic Examination, SSUrDNA assay and the present method can respectively detect the infection at the 3, 6, and 7 days post infection. The present method has a similar advantage with SSUrDNA assay of detecting the infection at early stage. However, the signal of SSUrDNA assay was saturated since the 7 days post infection (FIG. 2) and failed to distinguish the infection level thereafter. Whereas, the present method was capable to tell the difference of signal intensity from the data of the 7, 14, and 21 days post infection. That is, the molecular marker of the present invention can detect the infection level of the object to be examined and the application value thereof is higher than a SSUrDNA assay.

What is claimed is:

1. A molecular marker for detecting Nosema infection, comprising at least one sequence selected from a group consisting of DNA sequences of SR-22, SR-28, SR-71, and SR-85, cDNA sequences thereof, fragments thereof, and complementary sequences thereof.

2. The molecular marker of claim 1, comprising at least one sequence selected from a group consisting of SEQ ID NO 01, SEQ ID NO 02, SEQ ID NO 03, SEQ ID NO 04, SEQ ID NO 24, SEQ ID NO 25, SEQ ID NO 26, SEQ ID NO 27, SEQ ID NO 28, SEQ ID NO 29, SEQ ID NO 30, and SEQ ID NO 31.

3. The molecular marker of claim 1, further comprising a SSUrDNA sequence of Nosema.

4. A method for detecting Nosema infection, comprising:
(A) obtaining a genetic material;
(B) obtaining a nucleic acid, having at least parts of its sequences being complementary to the molecular marker of claim 1 or claim 2;
(C) base-pairing said nucleic acid with said genetic material and examining the result of said base-pairing.

5. The method of claim 4, wherein said genetic material is deoxyribonucleic acid, ribonucleic acid, or a combination thereof.

6. The method of claim 4, wherein said nucleic acid is deoxyribonucleic acid, ribonucleic acid, or a combination thereof.

7. The method of claim 4, wherein said nucleic acid is a primer set, a probe, or a combination thereof.

8. The method of claim 7, wherein said primer set comprising: a primer set of SEQ ID NO 05 and SEQ ID NO 06, a primer set of SEQ ID NO 07 and SEQ ID NO 08, a primer set of SEQ ID NO 09 and SEQ ID NO 10, or a primer set of SEQ ID NO 11 and SEQ ID NO 12.

9. The method of claim 4, wherein said step (c) is achieved by polymerase chain reaction, reverse-transcription polymerase chain reaction, real-time polymerase chain reaction, dot blotting, or a combination thereof.

10. The method of claim 4, comprising detecting the existence of SSUrDNA of Nosema in said genetic material.

11. The method of claim 4, wherein said detecting Nosema infection comprises detecting the infection level.

12. A kit for detecting Nosema infection, comprising:
a nucleic acid, having at least parts of its sequences being complementary to the molecular marker of claim 1.

13. The kit of claim 12, wherein said molecular marker comprises at least one sequence selected from a group consisting of SEQ ID NO 01, SEQ ID NO 02, SEQ ID NO 03, SEQ ID NO 04, SEQ ID NO 24, SEQ ID NO 25, SEQ ID NO 26, SEQ ID NO 27, SEQ ID NO 28, SEQ ID NO 29, SEQ ID NO 30, and SEQ ID NO 31.

14. The kit of claim 12, wherein said nucleic acid is deoxyribonucleic acid, ribonucleic acid, or a combination thereof.

15. The kit of claim 12, wherein said nucleic acid is a primer set, a probe, or a combination thereof.

16. The kit of claim 15, wherein said primer set comprising: a primer set of SEQ ID NO 05 and SEQ ID NO 06, a primer set of SEQ ID NO 07 and SEQ ID NO 08, a primer set of SEQ ID NO 09 and SEQ ID NO 10, or a primer set of SEQ ID NO 11 and SEQ ID NO 12.
